(19) United States
(12) Patent Application Publication
(10) Pub. No.: US 2004/0133240 A1
(43) Pub. Date: Jul. 8, 2004

(54) Electrotherapy system, device, and method for treatment of cardiac valve dysfunction

(75) Inventors: John M. Adams, Sammamish, WA (US); Scott J. Wolf, Bellevue, WA (US); David Reuter, Bothell, WA (US); Mark Mathis, Kirkland, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: Cardiac Dimensions, Inc.

(21) Appl. No.: 10/338,081

(22) Filed: Jan. 7, 2003

(51) Int. Cl. A61N 1/36

(52) U.S. Cl. 607/2

(57) ABSTRACT

A system for treating cardiac valve dysfunction includes a lead with electrodes in electrical communication with muscle tissue proximate to a cardiac valve to be treated. Electrical energy is delivered to the lead electrodes to stimulate contraction of the muscle tissue and thereby constrict the cardiac valve. The lead may be received within a blood vessel in the patient. Detection circuitry may detect a physiological signal in the patient for controlling the timing of delivery of electrical energy. The lead may have one or more undulations. The lead may also be combined with a prosthesis to provide a combined electromechanical cardiac valve therapy. The lead can be attached to the prosthesis or formed integrally with the prosthesis. One embodiment implanted in the coronary sinus is used to treat dilated cardiomyopathy of the mitral valve.

### FIELD OF THE INVENTION

The present invention relates generally to methods and apparatus for treatment of cardiac dysfunction, and more specifically to treatment of cardiac valve dysfunction.

### BACKGROUND OF THE INVENTION

A mammalian heart typically includes multiple chambers through which blood is pumped into the circulatory system. A human heart includes four chambers, namely, a left and right atrium and a left and right ventricle. Blood is first pumped from the atria to the ventricles during atrial contraction. During ventricular contraction, blood is pumped from the ventricles into the circulatory system of the body.

Valves separate the various chambers of a mammalian heart and control the direction of blood flow through the heart. In a human patient, for example, the left atrium is separated from the left ventricle by the mitral valve. A normally functioning mitral valve permits blood to flow from the left atrium to the left ventricle, but not vice versa. Leaflets, or cusps, that form the mitral valve close upon one another when blood is pumped from the left ventricle to the circulatory system. This closure of the mitral valve prevents backflow or regurgitation of blood into the left atrium during ventricular contraction. A normally functioning mitral valve can withstand substantial back pressure of blood when the left ventricle contracts.

In a diseased or dysfunctioning state, the mitral valve leaflets may not fully close during ventricular contraction, and thus permit blood to leak from the left ventricle back into the left atrium. When this occurs, the blood flow to the body, i.e., cardiac output, is decreased. In response, the heart pumps harder in an effort to compensate for the decreased blood flow to the body.

Cardiac valve dysfunction may result from any number of disorders that weaken or damage the valve. For instance, rheumatic heart disease can cause thickening, rigidity, and retraction of the mitral valve leaflets. Other disorders, such as heart failure, atherosclerosis, hypertension, ventricle enlargement, connective tissue disorders, other congenital defects, endocarditis, and cardiac tumors may result in mitral valve dysfunction. In some instances, mitral valve prolapse may develop, which involves weakening and ballooning of the valve. Often, outward symptoms of mitral valve regurgitation in a patient are not observable, and when symptoms, such as fatigue, cough, palpitations, and shortness of breath do occur, they often develop gradually. Typically, cardiac valve dysfunction is detected by an echocardiogram.

Prior art methods of treating cardiac valve dysfunction, such as mitral valve regurgitation, require significant invasive procedures in the patient. In some cases, such procedures involve surgical techniques that repair the shape of the valve, including annuloplasty, which surgically restricts the valve annulus to reduce dilation of the valve. In this procedure, an annular or partially annular prosthesis may be secured inside the valve around the base of the valve leaflets to maintain the shape of the valve annulus during the opening and closing of the valve.

In other cases, complete replacement of the valve may be performed, particularly when a portion or all of the valve is seriously damaged or deformed. Replacement of a cardiac valve involves significant invasive procedures into the heart of the patient. These procedures are expensive and may entail substantial risk to the patient. Furthermore, the efficacy of the procedure may not be known until after the procedure is completed, at a time when it is difficult to make any adjustments in the treatment.

In another arena, cardiac electrotherapy has been developed for purposes of treating cardiac arrhythmias. For instance, electrical pulses from an implanted pacemaker can help a heart maintain a regular heartbeat. Defibrillation devices are known to electrically stimulate a heart to stop fibrillation and allow the heart to return to a normal sinus rhythm. While electrotherapy is known for treating cardiac arrhythmias, electrotherapy has not been considered for possible effect on cardiac valvular function. The present invention overcomes the above-noted deficiencies in the prior art and provides a method and apparatus for treating valvular dysfunction through electrotherapy.

### SUMMARY OF THE INVENTION

In one aspect, the present invention provides a system for treating cardiac valve dysfunction. The system includes a lead with electrodes configured for implantation in a patient such that the electrodes are in electrical communication with muscle tissue proximate to a cardiac valve to be treated. Stimulation circuitry controlled by control circuitry delivers electrical energy to the lead electrodes to stimulate contraction of the muscle tissue.

The lead may be configured to be received within a blood vessel in the patient. In one embodiment, the blood vessel is the coronary sinus which is positioned next to muscle tissue around the mitral valve of the patient.

A system constructed according to the invention may further include detection circuitry with electrodes configured to detect an electrical complex in the patient's heart. The control circuitry may control the delivery of electrical energy from the stimulation circuitry based on the detection of an electrical complex. For example, the detection circuitry may be configured to detect a P-wave in the patient's heart and produce a control signal signifying detection of a P-wave. Electrical energy may be delivered to the patient at a time determined based on the control signal, for example, a time following atrial contraction but preceding or coinciding with ventricular contraction.

In one aspect, the lead may have one or more undulations, either before or after implantation in the patient, placing the electrodes toward the muscle tissue that is proximate to the cardiac valve to be treated. In another aspect, the lead may be combined with a prosthesis. The lead can be attached to the prosthesis or formed integrally with the prosthesis. The prosthesis may be formed to exert a mechanical pressure toward the cardiac valve to be treated, while at the same time deliver electrical therapy to the valve via the lead electrodes.

In another embodiment, the invention provides a cardiac valve constricting device that includes electrodes configured for implantation in a patient. Again, the electrodes are placed in electrical communication with muscle tissue that is proximate to a cardiac valve to be constricted. A detector is configured to receive a physiological signal from the patient and, based on the physiological signal, produce a detection signal signifying detection of a contraction in the patient's heart. A stimulator receives the detection signal and delivers electrical energy to the electrodes in time relation to the detection signal to cause the muscle tissue to contract and exert a constricting pressure on the cardiac valve. The stimulator may be configured to deliver the electrical energy prior to or concurrent with another contraction in the patient's heart following the receipt of the detection signal.

In yet another embodiment, the invention provides an electrical lead for treatment of cardiac valve dysfunction. The lead may be constructed of a length of electrically conductive material in an insulating substrate, and a number of electrodes. The electrically conductive material is preferably configured for implementation in a patient such that the electrodes are in electrical communication with muscle tissue proximate to a cardiac valve to be treated. Electrical energy may be used to cause the muscle tissue to contract and exert a constricting pressure on the cardiac valve.

Still another embodiment is a device for treating dilated cardiomyopathy of the mitral valve in a patient's heart. The device may include a lead with electrodes configured for implantation in the coronary sinus of the patient's heart, and a stimulator. The stimulator delivers electrical energy to the electrodes and stimulates contraction of muscle tissue proximate to the coronary sinus. When the muscle tissue contracts, it causes the mitral valve to constrict. The stimulator may be configured to deliver electrical energy at a time determined based on a control signal signifying detection of an atrial contraction. The stimulator is preferably configured to deliver electrical energy to the lead electrodes prior to or concurrent with a ventricular contraction.

A combined electromechanical therapy system for treatment of cardiac valve dysfunction include an elongate member configured for implantation in a patient's heart to partially encircle the cardiac valve to be treated and an electrical lead connected to the elongate member. In this embodiment, the lead may have electrodes that deliver electrical energy to muscle tissue proximate to the cardiac valve when the elongate member is placed adjacent to the cardiac valve. A stimulator may deliver electrical energy to the electrodes to stimulate contraction of the muscle tissue and thereby exert a constricting pressure on the cardiac valve.

The elongate member may be configured to exert an inward constricting pressure on the cardiac valve when the member is placed adjacent to the cardiac valve. In one embodiment, the elongate member is attached to the muscle tissue proximate to the cardiac valve. In another embodiment, the elongate member and electrical lead are configured to be received within a blood vessel, such as a coronary sinus, next to the muscle tissue and the cardiac valve to be treated. In the latter embodiment, the elongate member may be formed with a “V” shaped end portion having a leg that exerts a pressure toward a wall of the blood vessel, resulting in an opposite inward pressure directed toward the cardiac valve.

Methods for treating cardiac valve dysfunction are also provided. One method includes implanting a lead with electrodes in a patient such that the electrodes are in electrical communication with muscle tissue proximate to a cardiac valve to be treated. Electrical energy is delivered to the lead electrodes to stimulate contraction of the muscle tissue, and thereby exert a constricting pressure on the cardiac valve. The methods may further include detecting an electrical complex in the patient's heart and delivering the electrical energy to the lead electrodes based on the detection of an electrical complex.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a pictorial diagram of an implantable electrotherapy device constructed in accordance with one exemplary embodiment of the present invention;

FIG. 2 is a top view of a human heart with the left atrium removed and the right atrium partially removed and further depicting electrical leads implanted in the heart in accordance with one exemplary embodiment of the present invention;

FIG. 3 is a block diagram of internal components used in the electrotherapy device shown in FIG. 1;

FIG. 4 is a circuit diagram illustrating electrical connections within an electrical lead constructed in accordance with one exemplary embodiment of the present invention; and

FIG. 5 is a graph illustrating P-wave detection and stimulus of the mitral valve annulus in accordance with an exemplary embodiment of the present invention.

FIG. 6 is a view of a human heart in which a portion of the atria has been removed, showing electrical leads implanted in the heart in accordance with one embodiment of the present invention wherein an arch-shaped prosthesis is also provided.

### DETAILED DESCRIPTION OF THE PREFERRED EMBODIMENT

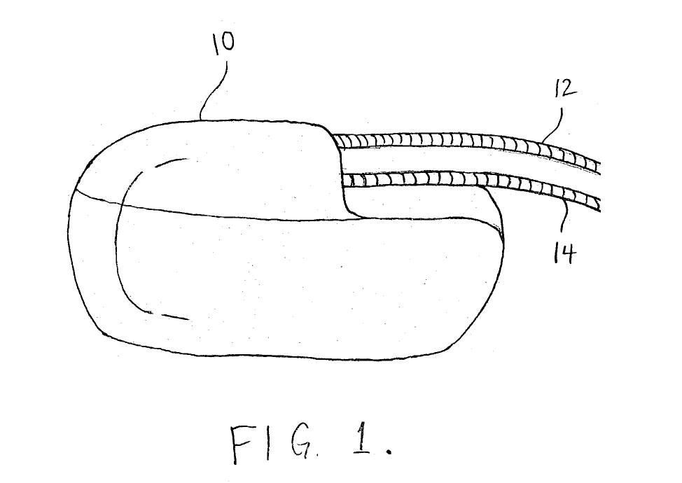

An exemplary embodiment of the present invention includes an implantable device 10, as shown in FIG. 1, to which electrical leads 12 and 14 are connected. The implantable device 10 is constructed to provide electrotherapy to cardiac tissue surrounding a valve in a heart. As will be understood from the description herein, the electrical stimulus provided by the device 10 is intended to cause the muscle cells surrounding the cardiac valve to contract in a manner that helps the cardiac valve maintain proper form during cardiac contraction and prevents regurgitation of blood through the valve. The electrotherapy device 10, as described herein, is particularly suited for stimulating muscle cells attached to the annulus that surrounds the mitral valve, though the invention is applicable to conditions in which other cardiac tissue is stimulated to maintain the proper shape of other valves.

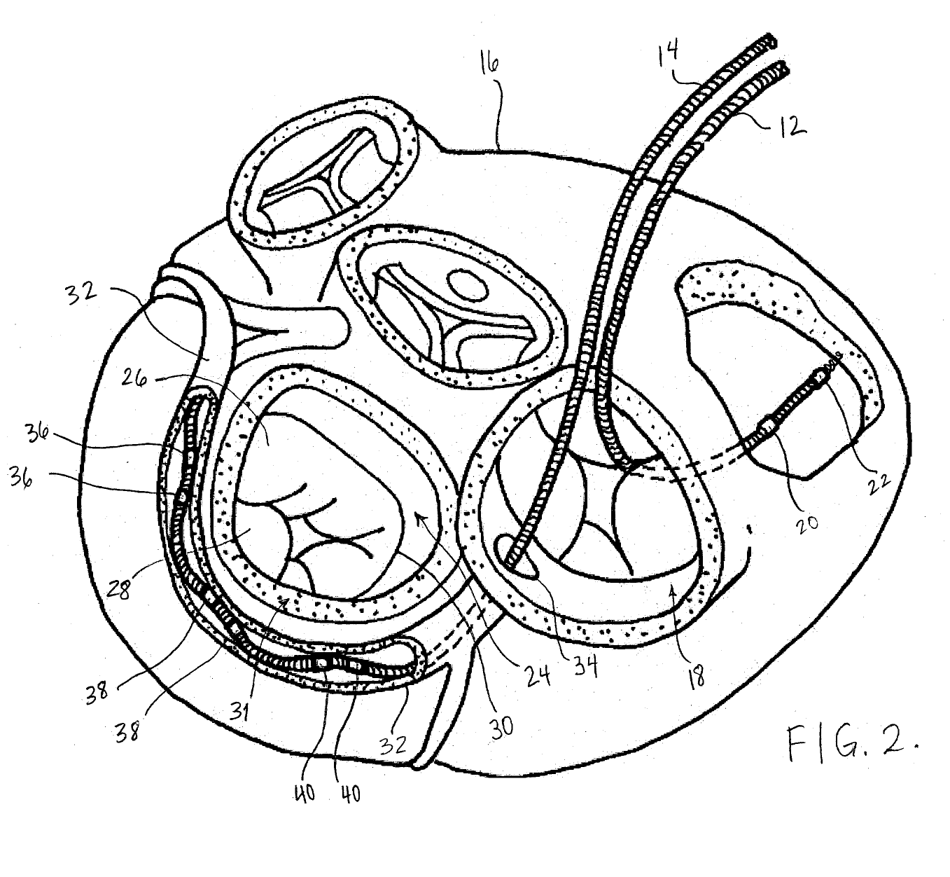

In one embodiment of the invention, the electrical leads 12 and 14 are introduced into a patient's heart 16, as illustrated in FIG. 2. The electrical leads 12 and 14 are preferably comprised of a length of electrically conductive material disposed within an insulating substrate. A plurality of electrodes are preferably attached to the insulating substrate and electrically connected to the conductive material. Prior to discussing further detail of the electrical leads 12 and 14, it is worthwhile to first observe the various characteristics of the heart 16 depicted in FIG. 2. FIG. 2 is a top view of a human heart in which the left atrium of the heart has been removed, thus exposing the mitral valve 24 that connects the left atrium to the left ventricle. The mitral valve 24 includes an anterior cusp 26 and a posterior cusp 28 attached to an annulus 30. The annulus 30 is comprised of a ring of collagen tissue. Surrounding the mitral valve annulus 30 is muscle tissue 31, in this instance comprised principally of atrial muscle tissue. The valve cusps 26 and 28 are anchored to the muscle wall of the heart by fibrous chords within the left ventricle (not shown) that support the cusps during contraction of the left ventricle. In a normally functioning mitral valve 24, the cusps 26 and 28 overlie each other during left ventricular contraction to prevent backflow of blood from the left ventricle to the left atrium. When the shape of the mitral valve 24 is distorted by disease or other dysfunction, insufficient closure of the valve cusps 26 and 28 leads to regurgitation of blood through the valve back into the left atrium.

In the embodiment shown in FIG. 2, the electrical leads 12 and 14 enter the heart through the superior vena cava into the right atrium 18 of the heart. The electrical lead 12 is preferably secured to cardiac tissue in a wall of the right atrium 18. The electrical lead 12 includes a pair of electrodes 20 and 22 configured to detect the occurrence of an electrical complex, such as a P-wave, in the patient's heart. As is well known in the art, a P-wave signals the contraction of the atria. The electrotherapy device 10 uses the detection of P-waves to synchronize the delivery of electrical stimuli to the heart, as discussed herein. For the purposes of implementing this embodiment of the invention, the electrical lead 12 may be comprised of a commercially available bipolar lead, the structure and function of which is well known in the art for detection of electrical complexes, including P-waves, when implanted in an atrium of the heart.

The embodiment of the invention shown in FIG. 2 recognizes that the coronary sinus 32 of the heart 16 is located proximate to and at least partially surrounds the mitral valve annulus 30. The coronary sinus 32, in this regard, includes the coronary sinus and/or the great cardiac vein. As will be understood from the description herein, electrical stimulation of the muscle tissue 31 near the annulus 30 helps maintain the shape of the mitral valve 24 and provide proper closure to the mitral valve cusps 26 and 28 during left ventricular contraction.

The electrical lead 14 is configured to be received within a blood vessel that is proximate to the cardiac valve to be treated. In FIG. 2, the electrical lead 14 is shown inserted into the coronary sinus 32 through the coronary sinus ostium 34. The electrical lead 14 includes at least one pair of electrodes that is used to stimulate the muscle tissue 31 in the area of the mitral valve annulus 30. For wider application of the stimulus, the electrical lead 14 is shown in FIG. 2 with multiple pairs of electrodes 36, 38, and 40. Where multiple pairs of electrodes are used, as shown, the electrical lead 14 preferably extends through a portion or all of the coronary sinus 32 that surrounds the mitral valve annulus 30. The electrode pairs 36, 38, and 40 may be spaced along the length of the electrical lead 14, as desired.

The electrical lead 14 may optionally include undulations in the lead that direct the electrode pairs 36, 38, and 40 against the wall of the coronary sinus 32, thus improving the conduction of the electrical stimulation from the electrode pairs through the muscle tissue 31 around the mitral valve annulus 30. Where the undulations are preformed in the lead, the electrical lead 14 should be flexible enough to be inserted into the coronary sinus 32 without damaging the coronary sinus wall, but rigid enough to maintain the undulations that place the electrode pairs 36, 38, and 40 against the coronary sinus wall. While electrode pairs are shown in the electrical lead 14, those having ordinary skill in the art will recognize other suitable electrode configurations for use with the invention, including an electrical lead 14 with single unipolar electrodes spaced along the length of the lead.

The electrodes 36, 38, and 40 deliver electrical energy that stimulates contraction of the muscle tissue 31 near the annulus 30. As the muscle tissue 31 contracts from the stimuli, it imparts an inward tension on the annulus 30 and helps pull the cusps 26 and 28 of the mitral valve together. By causing the muscle tissue 31 to contract slightly before or at the time the left ventricle contracts, the mitral valve cusps 26 and 28 are brought together and achieve proper closure during ventricular contraction.

FIG. 3 is a block diagram depicting various components that may be used in the electrotherapy device 10 shown in FIG. 1. The depiction of components in FIG. 3 is not intended to limit the configuration of the device 10 or limit the scope of the invention in any way. Other embodiments of the device 10 may include fewer or greater number of components in different configurations than that shown in FIG. 3.

As noted in FIG. 2, the electrical lead 12 includes electrodes 20 and 22 that are configured to detect electrical activity in the right atrium. The electrical signal detected by the electrodes 20 and 22 is delivered to an amplifier 42 shown in FIG. 3. The construction of amplifiers for amplifying electrical signals is well known in the art, and details of such are not required herein for one having ordinary skill in the art to practice the invention. The amplified signal that is output from the amplifier 42 is digitized in an analog-to-digital (A/D) converter 44. The digitized electrical signal output from the A/D converter 44 is delivered to detection circuitry that detects an electrical complex in the signal, in this case a P-wave detector 46. In this embodiment of the invention, the P-wave detector is implemented in a processor 48 and may be comprised of computer program instructions that cause the processor 48 to receive and analyze the digitized electrical signal for the occurrence of a P-wave. The computer program instructions may be stored in a memory 50. Other embodiments of the invention may implement the P-wave detector in separate, dedicated detection circuitry that uses hard wired or programmable components. In any event, the P-wave detector 46 monitors the electrical (electrogram) signal for peak values that represent P-waves in the patient's heart. The P-wave detector 46 is preferably configured to produce a control signal that indicates when a P-wave is observed in the electrical signal received from the electrodes 20 and 22. The control signal is preferably communicated to a stimulator 52, discussed in greater detail below. Although not shown, appropriate filters may be used on the electrical signal before and/or after the amplifier 42 or the A/D converter 44. The filters, implemented in hardware or software, may be used, for example, to attenuate noise, prevent aliasing, and/or emphasize those portions of the signal that are known to best reveal a P-wave when a P-wave occurs.

In this particular embodiment of the invention, the detection of P-waves in the right atrium triggers the delivery of electrical stimuli to the electrodes 36, 38, and 40 surrounding the mitral valve 24. In FIG. 3, the stimulator 52 is comprised of control circuitry that controls the delivery of the electrical stimuli. The stimulator 52 may include any type of circuitry capable of controlling the delivery of electrical energy to the electrical lead 14. As with the P-wave detector 46, the stimulator 52 may be implemented by program instructions stored in the memory 50 that cause the processor to output appropriate control signals. For example, the stimulator 52 may be a programmable pulse generator that directs the voltage, pulse width, and/or number of pulses to be delivered from stimulation circuitry, such as a driver circuit 54. The stimulator 52 controls the switching (on/off) of the driver circuit, as well as the voltage, to deliver the electrical pulses in the stimulus. Alternatively, the stimulator 52 may itself incorporate stimulation circuitry capable of delivering an electrical signal. In still other embodiments of the invention, the stimulator 52 may be implemented in separate, dedicated circuitry comprised of hard wired and/or programmable components. In FIG. 3, the control signals from the stimulator 52 are fed to the stimulation driver 54, which in turn produces the stimulus energy that is delivered to the electrical lead 14. The electrical lead 14 carries the electrical stimulus to the electrodes 36, 38, and 40 (FIG. 2). The driver 54 may obtain electrical energy from the same energy source, such as a long-life lithium battery (not shown), that powers the other components in the electrotherapy device 10.

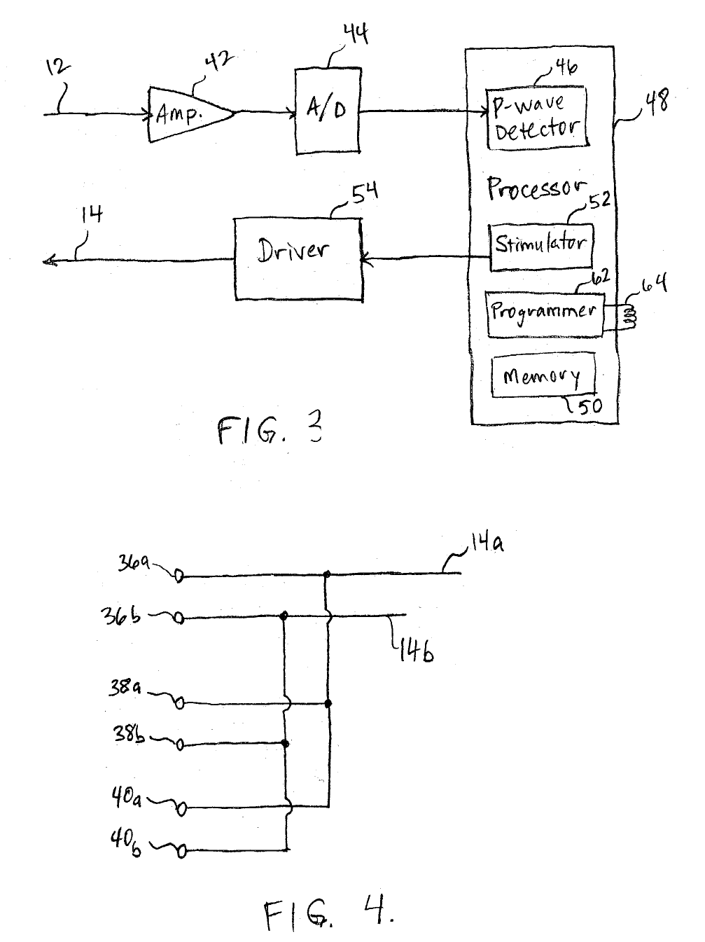

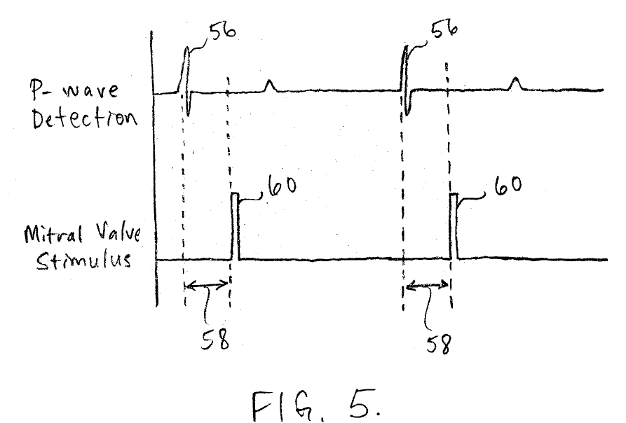

FIG. 4 is a circuit diagram illustrating a possible configuration for the electrical lead 14. In this configuration, the electrical lead 14 includes two electrically isolated conductors 14a and 14b. The pair of electrodes 36 shown in FIG. 2 is comprised of electrodes 36a and 36b. Similarly, the pair of electrodes 38 is comprised of electrodes 38a and 38b, while the pair of electrodes 40 is comprised of electrodes 40a and 40b. The electrical conductor 14a is thus connected to the electrodes 36a, 38a, and 40a. The electrical conductor 14b is connected to the electrodes 36b, 38b, and 40b. Electrical stimulus provided from the driver 54 (FIG. 3) may be carried by the circuit line 14a to the electrodes 36a, 38a, and 40a. The electrical energy is then conducted through the cardiac tissue surrounding the mitral valve, including the mitral valve annulus, and received by the electrodes 36b, 38b, and 40b, to complete the electrical circuit.

Those with ordinary skill in the art will recognize that the configuration of circuitry shown in FIG. 4 results in simultaneous delivery of the stimulus to the tissue surrounding the mitral valve annulus. In other embodiments of the invention, one or more delay elements may be introduced to cause a timed, cascading delivery of electrical energy to each of the electrode pairs 36, 38, and 40.

FIG. 5 is a graph that depicts possible timing between P-wave detection and delivery of the stimulus in one embodiment of the invention. The upper portion of FIG. 5 depicts atrial electrogram received via the electrical lead 12. At the time of atrial contraction, a sharp spike 56 in the electrical signal (i.e., a P-wave) is detected. The detection of a P-wave by the P-wave detector 46 may start a timer within the stimulator 52 that measures a time period 58, as shown in the lower portion of FIG. 5. For this exemplary embodiment, delays on the order of 5 ms to 75 ms may be appropriate. At the expiration of the time period 58, the stimulator 52 causes the driver 54 to output the electrical stimulus 60 that is delivered to the patient via the electrical lead 14. For this exemplary embodiment, the electrical stimulus 60 may be comprised of a pulse on the order of 3 to 15 volts at 0.5 to 1.0 ms duration. The electrical stimulus 60 is communicated to the muscle tissue 31 near the mitral valve annulus 30 causing the muscle cells to contract and thereby constrict the mitral valve 24. The length of the time period 58 may be determined from the delay between atrial contraction and ventricular contraction in the patient. The time of delivery of the mitral valve stimulus 60 is preferably at or slightly before ventricular contraction so that the mitral valve 24 is constricted when ventricular contraction occurs, thus reducing or eliminating regurgitation of blood.

As noted above, the operation of the P-wave detector 46 and stimulator 52 (FIG. 3) may be controlled by program instructions stored in the memory 50. The memory 50 may further include preprogrammed electrical characteristics, such as threshold peak values, that the P-wave detector uses to identify the occurrence of a P-wave. The memory 50 also may store information such as the time period 58 that the stimulator 52 uses to determine when the driver 54 should deliver the electrical stimulus. For ease of adjusting the operation of the electrotherapy device 10, the device may include a programmer 62 that receives and modifies information, including program instructions, in the memory 50. To enable the device 10 to be programmed after it is implanted in a patient, the programmer 62 is preferably connected to an antenna 64 that enables wireless communication with the device 10 through the patient. The construction of suitable communication components for transmitting and receiving information in an implantable device is well known in the art of pacing and does not require detailed discussion herein. Furthermore, while the programmer 62 is shown integrated within the processor 48 in (FIG. 3), those of ordinary skill in the art will appreciate that the programmer 62 may be implemented in separate circuitry that communicates with the memory 50.

The present invention thus provides an improved method and apparatus for treatment of cardiac valvular dysfunction. Although a preferred embodiment of the invention used for treating mitral valve regurgitation includes an electrical lead that is inserted into the coronary sinus of the patient, those of ordinary skill in the art will appreciate that the electrical lead 12 may be placed anywhere near the mitral valve annulus 30 such that electrical stimulus delivered to the lead causes the muscle cells in the annulus to constrict. Accordingly, the electrical lead may be positioned outside the coronary sinus in or near the mitral valve annulus. The preferred embodiment described above takes advantage of the coronary sinus because inserting the lead into the coronary sinus can be less invasive than other methods of bringing the electrical lead into contact with cardiac tissue next to the mitral valve annulus. Using the coronary sinus in this manner may also reduce the cost of the medical procedure needed to place the lead and provide electrotherapy to the mitral valve annulus.

In addition to modifying program instructions in the memory 50, the programmer 62 and antenna 64 also allow the stimulator 52 to be remotely adjusted while the patient's heart is monitored for regurgitation. The time period 58 may be shortened or lengthened, and/or the magnitude or shape of the pulse(s) delivered may be modified, while the patient's heart is beating, to optimally reduce or eliminate any valvular regurgitation. Again, the components and process of programming an electrical device implanted in a patient is common in the pacing art. One with ordinary skill in the art may adapt such components and procedure without undue effort for use with the present invention.

It will be further appreciated that the features of the device 10 depicted herein are intended to illustrate the operation of at least one preferred embodiment of the invention, and are not intended to limit the scope of the invention. The features of the invention may be integrated into other commercially available products, such as a conventional DDD pacemaker, an ICD, and/or a bi-ventricular pacemaker.

One significant advantage of the present invention is that the therapy provided to the patient does not rely on mechanical characteristics of a prosthesis to maintain the form and function of a cardiac valve during cardiac contraction. The contraction of the muscle tissue near the annulus of the valve may be regulated by the magnitude and timing of the electrical stimulus delivered to the electrical lead that is placed proximate to the annulus. Over time, as a patient's cardiac condition changes, the electrical stimulus may be modified to adapt to the patient's condition to provide optimal therapy to the cardiac valve.

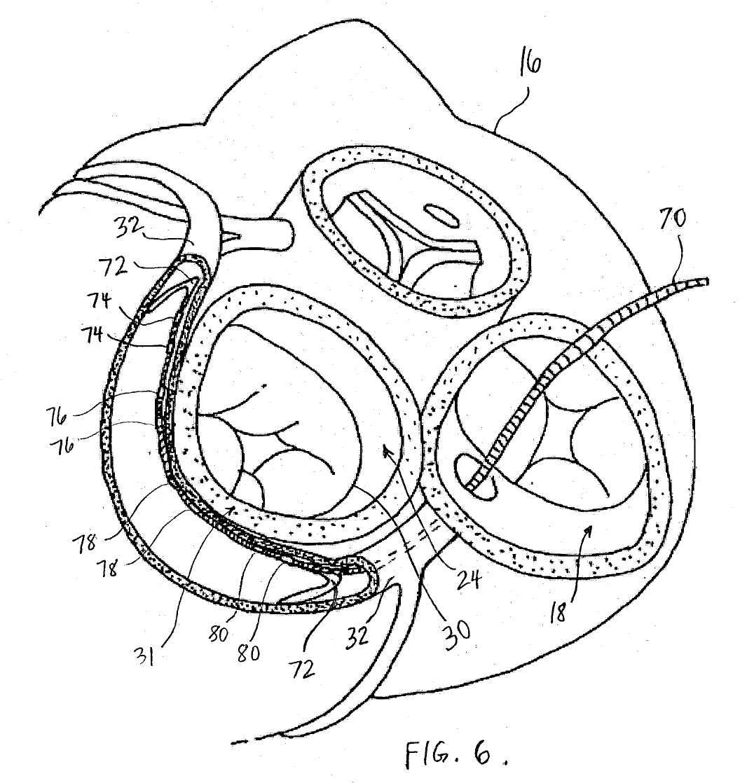

While the therapy provided by the present invention does not necessarily rely on mechanical characteristics of a prosthesis, an embodiment of the invention may advantageously be combined with a prosthesis that exerts physical pressure on the valve annulus. As noted previously herein, various prostheses have been proposed for treating cardiac valve dysfunction and generally include annular or partially annular devices that fit in or around the base of a valve. One proposed solution for the mitral valve has been to implant an elongated, arch-shaped member into the coronary sinus of the heart to partially encircle the mitral valve and exert an inward radial pressure on the mitral valve annulus. As shown in FIG. 6, the electrical lead provided by the present invention may advantageously be combined with a preformed member of this type to provide a combined mechanical and electrical-based therapy to the valve.

As with FIG. 2, FIG. 6 depicts a patient's heart 16 in which a portion of the atria has been removed to expose the mitral valve 24. The annulus 30 of the mitral valve 24 is surrounded by muscle tissue 31.

The embodiment depicted in FIG. 6 includes an electrical lead 70 that functions similar to the electrical lead 14 depicted in FIG. 2. The electrical lead 70 includes a plurality of electrode pairs 74, 76, 78, and 80. While the electrical lead 70 is shown with a plurality of electrode pairs, alternative embodiments may include only a single pair of electrodes. Further embodiments may also be comprised of single, unipolar electrodes spaced along the length of the lead 70.

The electrical lead 70 depicted in FIG. 6 is attached to an arch-shaped prosthesis 72 that exerts an inward mechanical pressure on the mitral valve annulus 30. The prosthesis 72 is preferably comprised of a material, such as Nitinol, a nickel titanium alloy, that is flexible yet retains a preformed shaped that results in an inward radial pressure on the valve annulus 30. In the particular embodiment shown in FIG. 6, the prosthesis 72 includes end portions that are bent back in a “V” shape. The electrical lead 70 and prosthesis 72 are shown inserted into the coronary sinus 32 through the coronary sinus ostium in the right atrium 18. One leg of each of the “V” shaped ends of the prosthesis 72 exerts an outward pressure on the outer wall of the coronary sinus 32. This outward pressure on the coronary sinus wall results in an opposite inward pressure being directed by the remaining portion of the prosthesis 72 toward the mitral valve annulus 30. Of course, those skilled in the art will recognize that other forms of the prosthesis 72 may be used with the present invention. For example, the prosthesis 72 may be attached at one or both ends to the coronary sinus wall in a manner that pulls the coronary sinus wall inward toward the mitral valve annulus 30. It is also possible for the electrical lead 70 to be attached to a prosthesis 72 that does not necessarily exert an inward pressure on the mitral valve annulus. In any regard, the electrical lead 70 is configured to deliver electrical stimulus to the muscle tissue 31 in a manner similar to the electrical lead 14 described and shown in FIG. 2.

The electrical stimulus may be delivered in time relation to the occurrence of a P-wave in the atrium so that the muscle tissue 31 is stimulated, and thus contracted, slightly before or at the time the ventricle contracts and blood in the ventricle presses against the mitral valve cusps. Although not shown, the embodiment depicted in FIG. 6 may include an electrical lead 12, as shown in FIG. 2, disposed within the right atrium 18 to detect the occurrence of a P-wave. The electrode pairs 74, 76, 78, and 80 may be spaced along the length of the electrical lead 70, as desired. Further, the electrical stimulus may be delivered simultaneously by the electrode pairs, or if desired, may be cascaded from one electrode pair to another. Again, the timing of the electrical stimulus preferably results in a muscle contraction around the valve annulus 30 causing the valve 24 to more fully close during ventricular contraction. The electrotherapy device delivering the electrical stimulus via the electrical lead 70 may be configured with components as shown and described in FIG. 3. By providing both a mechanical therapy and electrical therapy to the mitral valve 24, as shown in FIG. 6, more consistent closure of the mitral valve 24 may be optimally achieved.

While various embodiments of the invention have been illustrated and described, it will be appreciated that changes can be made therein without departing from the spirit and scope of the invention. The scope of the invention, therefore, should be determined from the following claims and equivalents thereto.

The embodiments of the invention in which an exclusive property or privilege is claimed are defined as follows:

1. A system for treating cardiac valve dysfunction, comprising:
(a) a lead with electrodes configured for implantation in a patient such that the electrodes are in electrical communication with muscle tissue proximate to a cardiac valve to be treated;
(b) stimulation circuitry in electrical communication with the lead for delivering electrical energy to the lead electrodes to stimulate contraction of the muscle tissue; and
(c) control circuitry in communication with the stimulation circuitry for controlling the delivery of the electrical energy to the lead electrodes.

2. The system of claim 1, in which the lead is configured to be received within a blood vessel in the patient.

3. The system of claim 2, in which the blood vessel is the coronary sinus positioned next to muscle tissue that is proximate to the mitral valve of the patient.

4. The system of claim 1, further comprising detection circuitry with electrodes configured to detect an electrical complex in the patient's heart, wherein the control circuitry is further configured to control the delivery of electrical energy based on the detection of an electrical complex.

5. The system of claim 4, in which the detection circuitry is configured to detect a P-wave in the patient's heart.

6. The system of claim 4, in which the detection circuitry further produces a control signal that is communicated to the control circuitry signifying detection of an electrical complex.

7. The system of claim 6, in which the control circuitry is configured to cause the stimulation circuitry to deliver the electrical energy at a time determined based on the control signal.

8. The system of claim 1, in which the lead is formed to have one or more undulations.

9. The system of claim 1, in which the lead has one or more undulations that place the electrodes toward the muscle tissue proximate to the cardiac valve to be treated when the lead is implanted in the patient.

10. The system of claim 1, in which the lead is combined with a prosthesis.

11. The system of claim 10, in which the lead is attached to the prosthesis.

12. The system of claim 10, in which the lead is formed integrally with the prosthesis.

13. The system of claim 10, in which the prosthesis is formed to exert a mechanical pressure directed toward the cardiac valve to be treated.

14. A cardiac valve constricting device, comprising:
(a) electrodes configured for implantation in a patient such that the electrodes are in electrical communication with muscle tissue that is proximate to a cardiac valve to be constricted;
(b) a detector configured to receive a physiological signal from the patient and, based on the physiological signal, produce a detection signal signifying detection of a contraction in the patient's heart; and
(c) a stimulator configured to receive the detection signal and deliver electrical energy to the electrodes in time relation to the detection signal to cause the muscle tissue to contract and exert a constricting pressure on the cardiac valve.

15. The cardiac valve constricting device of claim 14, in which the physiological signal is an electrogram signal detected in the patient's heart.

16. The cardiac valve constricting device of claim 15, in which the detector is configured to detect a P-wave in the patient's heart.

17. The cardiac valve constricting device of claim 14, in which the stimulator is configured to deliver the electrical energy prior to or concurrent with another contraction in the patient's heart following the receipt of the detection signal.

18. The cardiac valve constricting device of claim 17, in which the detection signal signifies detection of an atrial contraction and the electrical energy is delivered prior to or concurrent with a ventricular contraction.

19. The cardiac valve constricting device of claim 14, in which the lead is configured to be received within a blood vessel in the patient.

20. The cardiac valve constricting device of claim 19, in which the blood vessel is the coronary sinus that places the electrodes next to muscle tissue that is proximate to the mitral valve of the patient.

21. An electrical lead for treatment of cardiac valve dysfunction, comprising:
(a) a length of electrically conductive material in an insulating substrate; and
(b) a plurality of electrodes electrically connected to the conductive material;
in which the electrically conductive material is configured for implementation in a patient such that the plurality of electrodes are in electrical communication with muscle tissue proximate to a cardiac valve to be treated, the plurality of electrodes being configured to deliver electrical energy to the muscle tissue to cause the muscle tissue to contract and exert a constricting pressure on the cardiac valve.

22. The electrical lead of claim 21, in which the lead is formed with one or more undulations.

23. The electrical lead of claim 21, in which the lead has one or more undulations that place the plurality of electrodes in a position toward the muscle tissue that is proximate to the cardiac valve to be treated.

24. The electrical lead of claim 21, in which the lead is combined with a prosthesis.

25. The electrical lead of claim 24, in which the lead is attached to the prosthesis.

26. The electrical lead of claim 24, in which the electrical lead is formed integrally with the prosthesis.

27. The electrical lead of claim 24, in which the prosthesis is configured to exert a mechanical pressure directed toward the cardiac valve to be treated when the electrical lead and prosthesis are implanted in the patient.

28. A device for treating dilated cardiomyopathy of the mitral valve in a patient's heart, comprising:
(a) a lead with electrodes configured for implantation in the coronary sinus of the patient's heart; and
(b) a stimulator in electrical communication with the lead electrodes for delivering electrical energy to the electrodes and stimulating contraction of muscle tissue proximate to the coronary sinus that causes the mitral valve to constrict.

29. The device of claim 28, further comprising a detector configured to detect a P-wave in the patient's heart.

30. The device of claim 29, in which the detector is further configured to produce a control signal that is communicated to the stimulator to signify detection of a P-wave.

31. The device of claim 30, in which the stimulator is configured to deliver electrical energy at a time determined based on the control signal received from the detector.

32. The device of claim 30, in which the control signal signifies detection of an atrial contraction and the stimulator is configured to deliver electrical energy to the lead electrodes prior to or concurrent with a ventricular contraction.

33. The device of claim 28, in which the lead has one or more undulations that place the electrodes toward the muscle tissue proximate to the mitral valve.

34. The device of claim 28, further comprising a prosthesis configured for implantation in the coronary sinus with the lead to exert constricting mechanical pressure on the mitral valve.

35. A combined electromechanical therapy system for treatment of cardiac valve dysfunction, comprising:
(a) an elongate member configured for implantation in a patient's heart to partially encircle the cardiac valve to be treated;
(b) an electrical lead connected to the elongate member, the lead having a plurality of electrodes for delivering electrical energy to muscle tissue proximate to the cardiac valve when the elongate member is placed adjacent to the cardiac valve; and
(c) a stimulator in electrical communication with the lead electrodes for delivering electrical energy to the electrodes to stimulate contraction of the muscle tissue and thereby exert a constricting pressure on the cardiac valve.

36. The combined electromechanical therapy system of claim 35, in which the elongate member is further configured to exert an inward constricting pressure on the cardiac valve when the member is placed adjacent to the cardiac valve.

37. The combined electromechanical therapy system of claim 35, in which the elongate member is attached to the muscle tissue proximate to the cardiac valve.

38. The combined electromechanical therapy system of claim 35, in which the elongate member and electrical lead are configured to be received within a blood vessel next to the muscle tissue and the cardiac valve to be treated.

39. The combined electromechanical therapy system of claim 38, in which the elongate member and electrical lead are configured to be implanted in a coronary sinus of the patient proximate to a mitral valve of the patient.

40. The combined electromechanical therapy system of claim 38, in which at least one end of the elongate member has a “V” shaped end portion having a leg that exerts a pressure toward a wall of the blood vessel resulting in an opposite inward pressure being directed toward the cardiac valve.

41. The combined electromechanical therapy system of claim 35, further comprising a detector configured to detect an electrical complex in the patient's heart.

42. The combined electromechanical therapy system of claim 41, in which the electrical complex is a P-wave.

43. The combined electromechanical therapy system of claim 42, in which the detector is further configured to produce a control signal that is communicated to the stimulator to signify detection of a P-wave.

44. The combined electromechanical therapy system of claim 43, in which the stimulator is configured to deliver the electrical energy to the lead electrodes at a time determined based on the control signal.

45. The combined electromechanical therapy system of claim 35, in which the electrical lead is integrated with the elongate member.

46. A method for treating cardiac valve dysfunction, comprising:
(a) implanting a lead with electrodes in a patient such that the electrodes are in electrical communication with muscle tissue proximate to a cardiac valve to be treated; and
(b) delivering electrical energy to the lead electrodes to stimulate contraction of the muscle tissue, and thereby exert a constricting pressure on the cardiac valve.

47. The method of claim 46, further comprising detecting an electrical complex in the patient's heart and delivering the electrical energy to the lead electrodes based on the detection of an electrical complex.

48. The method of claim 47, in which the electrical complex is a P-wave.

49. The method of claim 47, further comprising producing a control signal signifying detection of an electrical complex.

50. The method of claim 49, further comprising delivering electrical energy to the lead electrodes at a time determined based on the control signal.

51. The method of claim 46, in which the lead is implanted within a blood vessel in the patient.

52. The method of claim 51, in which the lead is implanted in the coronary sinus of the patient proximate to the patient's mitral valve.

53. The method of claim 46, further comprising forming undulations in the lead.

54. The method of claim 53, in which the undulations are formed to place the lead electrodes toward the muscle tissue proximate to the cardiac valve to be treated.

55. The method of claim 46, further comprising combining the lead with a prosthesis.

56. The method of claim 55, further comprising implanting the prosthesis in the patient such that the prosthesis exerts a mechanical pressure toward the cardiac valve to be treated.
